(19) United States
(12) Patent Application Publication
(10) Pub. No.: US 2009/0233891 A1
(43) Pub. Date: Sep. 17, 2009

(54) Pharmaceutical lotion comprising fluticasone propionate

(75) Inventors: Nilendu Sen, Mumbai (IN); Kusum Gole, Maharashtra (IN); Manoj Soni, Mumbai (IN)

Correspondence Address: GLENMARK PHARMACEUTICALS INC USA, 750 CORPORATE DRIVE, MAHWAH, NJ, 07430, US

(73) Assignee: GLENMARK GENERICS LTD., Mumbai (IN)

(21) Appl. No.: 12/400,886

(22) Filed: Mar. 10, 2009

Related U.S. Application Data
(60) Provisional application No. 61/134,276, filed on Jul. 8, 2008.

(30) Foreign Application Priority Data
Mar. 11, 2008 (IN) 494/MUM/2008

(51) Int. Cl. A61K 31/56; A61P 17/00

(52) U.S. Cl. 514/178

(57) ABSTRACT

The present invention generally relates to a pharmaceutical composition. More particularly, the present invention relates to a stable topical lotion comprising at least a therapeutically effective amount of an androstane steroid compound or a pharmaceutically acceptable salt or ester thereof and one or more occlusive agents.

### PRIORITY

This application claims the benefit under 35 U.S.C. §119 to Indian Provisional Application No. 494/MUM/2008, filed on Mar. 11, 2008 and to U.S. Provisional Application No. 61/134,276, filed Jul. 8, 2008, the contents of each of which, are hereby incorporated by reference.

### BACKGROUND OF INVENTION

1. Technical Field

The present invention generally relates to a stable topical lotion comprising at least a therapeutically effective amount of fluticasone or a pharmaceutically acceptable salt or ester thereof and one or more occlusive agents.

2. Description of the Related Art

Fluticasone propionate [also known as S-(fluoromethyl) 6a, 9-difluoro-11β, 17-dihydroxy-16a-methyl-3-oxoandrosta-1,4-diene-17β-carbothioate, 17-propionate] described as an anti-inflammatory steroid in U.S. Pat. No. 4,335,121, is shown below:

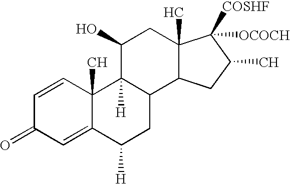

Fluticasone propionate is a white to off-white powder with a molecular weight of 500.6, and the empirical formula is C₂₅H₃₁F₃O₅S. It is practically insoluble in water, freely soluble in dimethyl sulfoxide and dimethylformamide, and slightly soluble in methanol and 95% ethanol. Fluticasone propionate is a synthetic corticosteroid, which is related to the naturally-occurring steroid hormone, cortisol (hydrocortisone), produced by the adrenal glands. Fluticasone propionate is known as a potent agent for the treatment of inflammatory respiratory disorders such as asthma, perennial rhinitis and of topical inflammatory conditions.

Fluticasone propionate is marketed worldwide under the tradename of Cutivate®, as a cream, ointment and lotion.

### SUMMARY OF THE INVENTION

The present invention generally relates to a pharmaceutical composition provides a stable topical lotion comprising at least a therapeutically effective amount of an androstane steroid compound or a pharmaceutically acceptable salt or ester thereof and one or more occlusive agents.

More particularly, the present invention provides that the pharmaceutical composition is in the form of a stable topical lotion comprising at least a therapeutically effective amount of an androstane steroid compound or a pharmaceutically acceptable salt or ester thereof and one or more occlusive agents.

The present invention provides a stable topical lotion comprising at least a therapeutically effective amount of fluticasone or a pharmaceutically acceptable salt or ester thereof and one or more occlusive agents.

The present invention provides a stable topical lotion comprising (a) about 0.005 wt % to about 1.0 wt % fluticasone or a pharmaceutically acceptable salt or ester thereof; (b) one or more occlusive agents; and, (c) an emulsifying effective amount of a surfactant.

The present invention provides a stable topical lotion for the treatment of skin conditions (i.e., dermatological disorders) comprising (a) about 0.005 wt % to about 1.0 wt % fluticasone or a pharmaceutically acceptable salt or ester thereof; (b) about 1.0 wt % to about 10.0 wt % of one or more C₁₄ to C₂₀ fatty alcohols; (c) about 1.0 wt % to about 5.0 wt % of at least one skin conditioning agent; (d) about 5.0 wt % to about 15.0 wt % of propylene glycol; (e) about 5.0 wt % to about 15.0 wt % mineral oil or soft white paraffin, and the balance being water.

The present invention provides a topical lotion comprising (a) about 0.005 wt % to about 1.0 wt % fluticasone propionate; (b) about 3.0 wt % to about 7.0 wt % of one or more C₁₄ to C₂₀ fatty alcohols; (c) about 0.5 wt % to about 3.0 wt % of at least one skin conditioning agent; (d) about 0.25 wt % to about 3.0 wt % of at least one surfactant; (e) about 7.0 wt % to about 12.0 wt. % propylene glycol; (f) about 10.0 wt % to about 15.0 wt % mineral oil or soft white paraffin; and the balance is water, preferably USP purified water.

The present invention provides a method of treating a skin condition (i.e., dermatological disorder) comprising applying to the area of the skin in need of treatment thereof, a topical lotion comprising (a) about 0.005 wt % to about 1.0 wt % fluticasone or a pharmaceutically acceptable salt or ester thereof; (b) one or more occlusive agents; and, (c) an effective emulsifying amount of a surfactant.

### DETAILED DESCRIPTION OF THE PREFERRED EMBODIMENTS

The present invention is directed to a stable pharmaceutical composition comprising at least fluticasone or a pharmaceutically acceptable salt or ester thereof and one or more occlusive agents. The stable pharmaceutical composition preferably comprises a topical lotion, which is an oil-in-water emulsion.

U.S. Pat. No. 7,300,669 discloses that fluticasone lotion exhibit increased vasoconstrictor potency of fluticasone at decreased concentrations of occlusive agent, i.e., under 10 w/w %, thus increasing the steroid effectiveness. Albeit, it is recognized that the addition of an occlusive agent, such as mineral oil or soft paraffin, increases the vasoconstrictor potency of topical steroids, such as fluticasone; the downside is that the use of high concentrations of occlusive agents hi an oil-in-water emulsion formulation, can lead to instability of the O/W emulsion formulation and an unpleasant greasy topical feel to the skin.

The formulation, herein described, surprisingly shows an improved, more reliable topical formulation where the amount of active ingredient available for vasoconstrictor activity is increased and without requiring undue limitations on the compositions of the formulations. The formulation, herein described, surprisingly shows maximum occlusion of the active ingredient to increase the vasoconstrictor potency of topical steroids.

The formulation, herein described, surprisingly show unexpectedly that an occlusive agent such as mineral oil or soft paraffin can be incorporated into a dermatological composition of fluticasone or a pharmaceutically acceptable salt or ester thereof which can increase the vasoconstrictor activity or potency of the formulation. The preferred mineral oil is light mineral oil.

Fluticasone or a pharmaceutically acceptable salt or ester thereof preferably fluticasone proprionate, is present hi the composition in a concentration of from about 0.005 wt % to about 1.0 wt % and preferably about 0.005 wt % to about 0.5 wt %. An occlusive agent such as mineral oil or white soft paraffin are incorporated into the composition such as a lotion in an amount ranging from about 5.0 wt % to about 15.0 wt % and preferably from 10.0 wt % to about 15.0 wt %.

At least one conventional surfactant may be used in topical formulations to form the composition of the present invention. Suitable surfactants include, but are not limited to, polyoxyalkene oxides of one or more C₁₄ to C₂₀ fatty alcohols, polyoxyalkylene sorbitan esters, and the like and mixtures thereof. Preferred surfactants include Cetomacrogol®1000, Ceteth-20®, Tween® 40 or Brij® 78, Span® 20 and mixtures thereof preferably Cetomacrogol®. The surfactant may be present in the composition in a concentration ranging from about 0.25 wt % to about 3.0 wt %, preferably from about 0.5 wt % to about 2.0 wt %, and more preferably from about 0.75 wt % to about 1.5 wt %.

Skin conditioning agents include, but are not limited to, cholesterol, glycerine, glycerol monostearate, isopropyl myristate and palmitate, and lanolin alcohols, and the like and mixtures thereof, preferably, isopropyl myristate and cetostearyl alcohol. The skin conditioning agent is present in the composition in a concentration ranging from about 1.0 wt % to about 5.0 wt %, preferably from about 1.0 wt % to about 3.0 wt %, and more preferably from about 1.0 wt % to about 2.0 wt %. In a preferred embodiment, dimethicone is employed in connection with at least one skin conditioning agent. The concentration of dimethicone in the formulation may be up to at least about 5.0 wt %, preferably at least from about 0.5 wt % to about 3.0 wt % and more preferably from about at least 1.0% wt % to about at least 2.0 wt % of the lotion composition.

Propylene glycol may be present in the composition in a concentration ranging from about 5.0 wt % to about 15.0 wt %, preferably from about 7.0 wt % to about 12.0 wt. % and more preferably from about 9.0 wt % to about 11.0 wt %.

One or more fatty alcohol can be present in the formulation as a thickener and/or stabilizer. Useful fatty alcohols include C₁₄ to C₂₀ fatty alcohols. Representative examples include, but are not limited to, cetyl alcohol, stearyl alcohol, and cetostearyl alcohol, preferably, cetostearyl alcohol. The fatty alcohol is present in the composition in a concentration ranging from about 1.0 wt % to about 10.0 wt %, preferably from about 3.0 wt % to about 7.0 wt %, and more preferably from about 4.0 wt % to about 6.0 wt %.

Optionally, conventional preservatives may be used in the present invention. Preferably, preservatives employed in the formulation should pass U.S. Pharmacopoeia, British Pharmacopoeia and European Pharmacopoeia standards. Suitable preservatives include, but are not limited to, imidurea, methylparaben, propylparaben and the like, and mixtures thereof, preferably methylparaben and/or propylparaben.

The present invention provides a method comprising the administration of pharmaceutical compositions to humans, in need thereof, wherein the pharmaceutical composition comprises at least a therapeutically effective amount of fluticasone or a pharmaceutically acceptable salt or ester thereof and one or more occlusive agents.

A skin condition (or dermatological disorder) includes, but is not limited to, corticosteroid-responsive dermatosis, atropic dermatitis, inflammation, eczema, erythema, papulation, scaling, erosion, oozing, crusting and pruritis.

The present invention provides a method comprising the administration of pharmaceutical compositions to humans, in need thereof, wherein the pharmaceutical composition comprising at least a therapeutically effective amount of fluticasone or a pharmaceutically acceptable salt or ester thereof and one or more occlusive agents, is in the form of a topical lotion.

The present invention provides a method comprising the administration of pharmaceutical compositions to humans, in need thereof, wherein the pharmaceutical composition comprising at least a therapeutically effective amount of fluticasone or a pharmaceutically acceptable salt or ester thereof and one or more occlusive agents, is in the form of a topical lotion, preferably as an oil-in-water emulsion.

Treatment of skin conditions with the lotion of the present invention is accomplished by applying the composition to the affected areas to be treated. As one skilled in the art will readily appreciate, the treatment regimen varies from patient to patient depending on the condition to be treated. In general, the composition herein described, is applied once or twice a day to the area in need of treatment. Preferably, the composition, herein described, is used to treat atopic dermatitis, inflammatory and pruritic manifestations and corticosteroid-responsive dermatoses.

The composition herein described, can be manufactured in a conventional manner by mixing the ingredients at an elevated temperature (e.g., from about 45° C. to about 80° C., preferably from about 65° C. to about 70° C.) and then cooling the mixture to achieve a smooth, homogeneous oil-in-water emulsion.

The following examples merely illustrate the lotion compositions of the invention and are not to be construed as limiting the scope of the invention. Unless indicated otherwise, all weight percentages are based on the total weight of the composition.

### EXAMPLES

### Example 1

A topical lotion in accordance with the present invention was prepared having the following ingredients and amounts as set forth below in Table 1.

TABLE 1

| S. No. | Composition | % w/w |
|---|---|---|
| 1. | Fluticasone Propionate (Micronized) | 0.05 |
| 2. | Cetostearyl alcohol | 3.00 |
| 3. | Propylene Glycol | 10.0 |
| 4. | Cetomacrogol 1000 BP | 2.00 |
| 5. | Mineral oil light | 12.0 |
| 6. | Isopropyl myristate | 1.00 |
| 7. | Dimethicone 360 | 1.00 |
| 8. | Citric acid anhydrous | 0.05 |
| 9. | Sodium citrate anhydrous | 0.075 |
| 10. | Methylparaben | 0.17 |
| 11. | Propylparaben | 0.06 |
| 12. | Imidurea | 0.14 |
| 13. | Purified Water | q.s. |

Manufacturing Procedure

1. Drug suspension: Fluticasone propionate was dispersed in propylene glycol.

2. Oil phase: Methylparaben and propylparaben were added and dissolved to a molten mass of cetostearyl alcohol, cetomacrogol, mineral oil, isopropyl myristate, and dimethicone 360 and maintained at a temperature of 65° C. to 70° C.

3. Aqueous phase: Citric acid, sodium citrate and imidurea were added and dissolved in water. The solution was heated and maintained at a temperature 65° C. to 70° C.

4. Emulsification: The oil phase was added to the aqueous phase slowly and homogenized for 15 minutes. The drug suspension from 1 was added during homogenization.

5. Cooling: The product of 4 was allowed to cool under slow stirring. Adjusted water loss if necessary.

### Example 2

A topical lotion in accordance with the present invention was prepared having the following ingredients and amounts as set forth below in TABLE 2.

TABLE 2

| S. No. | Composition | % w/w |
|---|---|---|
| 1. | Fluticasone Propionate (Micronized) | 0.05 |
| 2. | Cetostearyl alcohol | 3.00 |
| 3. | Propylene Glycol | 10.00 |
| 4. | Cetomacrogol 1000 BP | 1.00 |
| 5. | Mineral oil | 12.0 |
| 6. | Sorbitan monostearate | 0.40 |
| 7. | Isopropyl myristate | 1.00 |
| 8. | Dimethicone 360 | 1.00 |
| 9. | Citric acid anhydrous | 0.046 |
| 10. | Sodium citrate anhydrous | 0.075 |
| 11. | Methylparaben | 0.17 |
| 12. | Propylparaben | 0.06 |
| 13. | Imidurea | 0.14 |
| 14. | Purified Water | q.s. |

Manufacturing Procedure

1. Drug suspension: Fluticasone propionate was dispersed in propylene glycol.

2. Oil phase: Methylparaben and propylparaben were added and dissolved to a molten mass of cetostearyl alcohol, cetomacrogol, mineral oil, isopropyl myristate, dimethicone 360 and maintained at temperature 65° C. to 70° C.

3. Aqueous phase: Citric acid, sodium citrate and imidurea were added and dissolved in water. The solution was heated and maintained at a temperature of 65° C. to 70° C.

4. Emulsification: The oil phase was added to the aqueous phase slowly and homogenized for 15 minutes. The drug suspension from step 1 was added during homogenization.

5. Cooling: The product of step 4 was allowed to cool under slow stirring. Adjusted water loss if necessary.

### Example 3

A topical lotion in accordance with the present invention was prepared having the following ingredients and amounts as set forth below in Table 3.

TABLE 3

| S. No. | Composition | % w/w |
|---|---|---|
| 1. | Fluticasone Propionate (Micronized) | 0.05 |
| 2. | Cetostearyl alcohol | 3.00 |
| 3. | Propylene Glycol | 10.00 |
| 4. | Cetomacrogol 1000 BP | 1.50 |
| 5. | Span 20 | 0.50 |
| 6. | Mineral oil | 5.00 |
| 7. | Isopropyl myristate | 1.00 |
| 8. | Dimethicone 360 | 1.00 |
| 9. | Citric acid anhydrous | 0.046 |
| 10. | Sodium citrate anhydrous | 0.075 |
| 11. | Methylparaben | 0.17 |
| 12. | Propylparaben | 0.06 |
| 13. | Imidurea | 0.14 |
| 14. | Purified Water | q.s. |

Manufacturing Procedure

1. Drug suspension: Fluticasone propionate was dispersed in propylene glycol.

2. Oil phase: Methylparaben and propylparaben were added and dissolved to a molten mass of cetostearyl alcohol, cetomacrogol, mineral oil, isopropyl myristate, dimethicone 360 and maintained at temperature 65° C. to 70° C.

3. Aqueous phase: Citric acid, sodium citrate and imidurea were added and dissolved in water. The solution was heated and maintained at a temperature of 65° C. to 70°.

4. Emulsification: The oil phase was added to the aqueous phase slowly and homogenized for 15 minutes. The drug suspension from 1 was added during homogenization.

5. Cooling: The product of 4 was allowed to cool under slow stirring. Adjusted water loss if necessary.

It will be understood that various modifications may be made to the embodiments disclosed herein. Therefore the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the claims appended hereto.

1. A stable topical lotion comprising about 0.005 wt % to about 1.0 wt % fluticasone or a pharmaceutically acceptable salt or ester thereof, an effective emulsifying amount of a surfactant and about 5.0 wt % to about 15.0 wt % one or more occlusive agents selected from soft paraffin or mineral oil.

2. A topical lotion for the treatment of skin conditions in humans, comprising (a) about 0.005 wt % to about 1.0 wt % fluticasone or a pharmaceutically acceptable salt or ester thereof; (b) about 1.0 wt % to about 10.0 wt % of one or more C₁₄ to C₂₀ fatty alcohols; (c) about 1.0 wt % to about 5.0 wt % of at least one skin conditioning agent; (d) about 5.0 wt % to about 15.0 wt % of propylene glycol; (e) about 5.0 wt % to about 15.0 wt % mineral oil or soft white paraffin, and the balance being water.

3. The lotion of claim 2, where the mineral oil is a light mineral oil.

4. A topical lotion for the treatment of skin conditions in humans, comprising (a) about 0.005 wt % to about 1.0 wt % fluticasone propionate; (b) about 3.0 wt % to about 7.0 wt % of one or more C₁₄ to C₂₀ fatty alcohols; (c) about 0.5 wt % to about 3.0 wt % of at least one skin conditioning agent; (d) about 0.25 wt % to about 3.0 wt % of at least one surfactant; (e) about 7.0 wt % to about 12.0 wt. % propylene glycol; (f) about 10.0 wt % to about 15.0 wt % mineral oil or soft white paraffin; and the balance is water, preferably USP purified water.

5. The lotion of claim 4, where the mineral oil is a light mineral oil.

6. A method of treating a skin condition in humans, comprising applying to the area of the skin in need of treatment thereof, a topical lotion comprising (a) about 0.005 wt % to about 1.0 wt % fluticasone or a pharmaceutically acceptable salt or ester thereof; (b) an effective emulsifying amount of a surfactant; and, (c) one or more occlusive agents selected from soft paraffin or mineral oil.
